(19) United States
(12) Patent Application Publication
(10) Pub. No.: US 2013/0312770 A1
(43) Pub. Date: Nov. 28, 2013

(54) SURGICAL DRAPE

(71) Applicant: Michael J. Young, Chicago, IL (US)

(72) Inventor: Michael J. Young, Chicago, IL (US)

(21) Appl. No.: 13/478,921

(22) Filed: May 23, 2012

(51) Int. Cl. A61B 19/08

(52) U.S. Cl. 128/853

(57) ABSTRACT

A surgical drape includes a first sheet of surgical drape material secured to a second sheet of surgical drape material by a removable adhesive disposed along an overlapping joint. A pre-defined work-through area is defined in a central portion of the surgical drape, and the overlapping joint intersects with the work-through. Optionally, removable areas of pre-defined size and shape around the work-through area are defined by frangible paths so that the work-through area can be selectively enlarged by tearing along one or more of the frangible paths to remove one or more of the removable areas. A tab facilitates gripping by a user for separating the first sheet from the second sheet. The work-through area may be a pre-cut opening or a pre-defined tear-away removable portion of the first and/or second sheets.

### FIELD OF THE INVENTION

The present invention relates generally to surgical drapes for use during surgical and/or other medical procedures.

### DESCRIPTION OF THE BACKGROUND

Surgical drapes are used in medical procedures, such as surgeries, to cover the skin surrounding an exposed surgical field on a patient. The surgical drape serves as a barrier to help maintain a sterile condition at the exposed surgical field.

In the past, surgical drapes were often performed by a doctor, nurse, or other practitioner at the site of the surgery using multiple sheets of sterilized or sanitized drape material. The practitioner typically taped or otherwise secured the individual pieces of drape material around the periphery of the surgical field. This conventional method of forming the surgical drape, however, typically wasted drape material, and proved inconvenient and time consuming to perform.

Additionally, the practitioner frequently needs to remove the surgical drape from the patient with one hand. For example, the practitioner typically secures a sterile dressing or a needle against the patient with one hand, while simultaneously removing the surgical drape with the other hand. This situation is particularly common when the practitioner is using a single drape having a cut-out center-hole of the type frequently used when performing certain procedures such as small-scale ophthalmic or plastic surgery, or when performing anesthetic applications. Often, a surgeon may have spent considerable time placing a specific dressing or obtaining vascular access. The surgeon must protect the surgical site, and not remove his/her hold-down hand while removing the sterile drape at the conclusion of the procedure. All of this must be performed while maintaining a sterile dressing and without disrupting any tubing connections. In such situations, surgeons find it difficult to remove a conventional taped-together surgical drape with one hand, while using the other hand to protect the surgical site from undesired movement or disruption. For example, when an epidural is inserted in the spine, or arterial lines are inserted in the wrist, surgeons typically use small drapes having a single hole. These small drapes are typically secured to the patient's skin using an adhesive. At the conclusion of the procedure, the practitioner must secure his/her work, and rip the drape off by tearing the adhesive away from skin. Many surgeons find this removal procedure to be quite cumbersome, and often find that the removal of the drape disturbs the dressings and/or lines.

The present inventor has developed a surgical drape that, in some arrangements, is believed to provide certain benefits and advantages not previously available from surgical drapes known heretofore.

### SUMMARY

A surgical drape is provided that includes a first sheet of surgical drape material secured to a second sheet of surgical drape material by a removable adhesive along an overlapping joint. A work-through area is defined in a central portion of the drape, and the overlapping joint intersects the work-through area. In this arrangement, the surgical drape may be easily removed from a patient by a single user with one hand while holding a needle, tube, or other object against the patient through the work-through area with the other hand.

In some aspects, the surgical drape may be provided as a kit in unassembled or partly assembled form.

Other aspects will become apparent upon consideration of the following detailed description and the appended claims.

### BRIEF DESCRIPTION OF THE DRAWINGS

FIG. 1 is top plan view of a surgical drape according to some aspects of the disclosure in an assembled condition; and

FIG. 2 is an isometric view of the surgical drape in a partially separated state.

### DETAILED DESCRIPTION

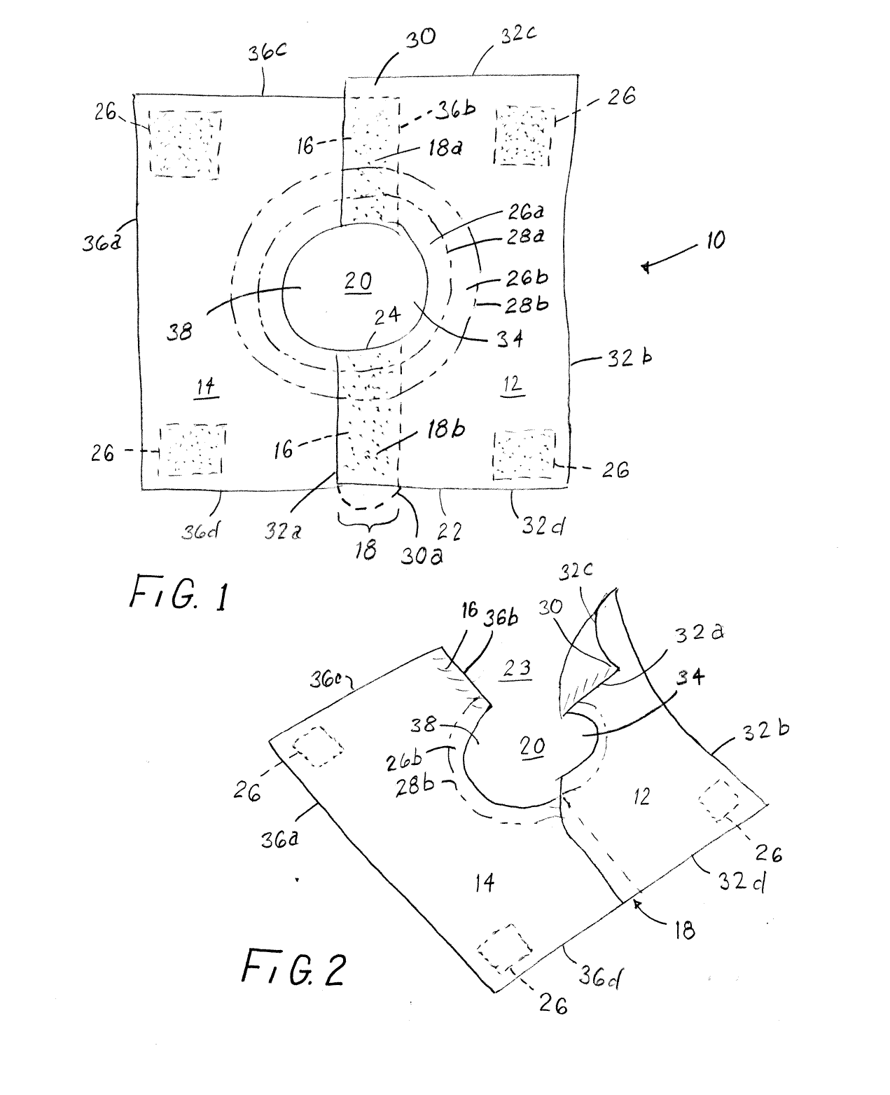

Turning now to FIG. 1, a surgical drape 10 according to some aspects of the invention includes a first sheet 12, a second sheet 14, an overlapping joint 18, and a work-through area 20 defined in a central portion of the drape. Each of the first and second sheets 12, 14 preferably is formed of a material approved and/or particularly well suited for use as a surgical drape. The overlapping joint 18 is defined by an area where the first sheet 12 overlaps the second sheet 14. A removable adhesive 16 is disposed along the overlapping joint 18 between the first and second sheets 12, 14 and secures the first sheet to the second sheet. An outer peripheral edge 22 of the surgical drape 10 is defined by the first and second sheets as secured together by the removable adhesive 16 along the overlapping joint 18. The work-through area 20 is spaced inwardly from the outer peripheral edge and preferably is pre-defined by a pre-cut opening through one or both of the first and second sheets 12, 14 and/or one or more removable areas, such as a tear-away portion of the first and/or second sheets defined by a frangible path of, for example, perforations surrounding the work-through area 20. The overlapping joint 18 intersects the work-through area 20 such that the first sheet 12 may be at least partly separated from the second sheet 14 by peeling the first sheet away from the second sheet along the overlapping joint and thereby opening a passageway 23 extending from the work-through area 20 to the outer peripheral edge 22.

Optionally, at least some portions of one side of the surgical drape 10, such as a back side as seen in FIG. 1, are covered with one or more additional areas of adhesive 26 to facilitate, for example, securing the surgical drape to the skin of a patient. (All directional modifiers used herein, such as left, right, up, down, front, back, etc., are solely used for ease of description with respect to the particular arrangement of the drawings, and are not to be construed as limiting the description to the particular directions indicated.) The adhesive 26 may cover the entire back side of the surgical drape 10 or may cover only portions, such as in the shape of dots, squares, lines, or other shapes. Also optionally, one or more removable areas 26a, 26b of pre-defined size and shape around the work-through area 20 are defined by one or more frangible paths 28a, 28b. The work-through area 20 can be selectively enlarged by tearing along one or more of the frangible paths 28a, 28b to remove one or more of the removable areas 26a, 26b away from the remaining portion of the surgical drape 10. A tab 30 optionally defined or carried by one of the sheets 12, 14 facilitates gripping by a user for separating the first sheet 12 from the second sheet 14.

As best seen in FIG. 2, the first sheet 12 may be separated from the second sheet 14 by peeling the first sheet 12 from the second sheet 14 along the overlapping joint 18, for example by pulling the first sheet 12 in the direction shown by arrow A. It is noted that in FIG. 2, the removable area 26a has been removed from the surgical drape 10, such as by tearing along frangible path 28a, to enlarge the work-through area 20. After the surgical drape 10 has been secured to a patient (not shown), by example with the spots of adhesive 26, a user, such as a surgeon, nurse, or other medical professional, can hold a needle or other instrument against the patient through the work-through area 20 with one hand and easily peel first sheet 12 away from the second sheet 14 and the patient with the other hand without disturbing the needle. Thereafter, the user may simply peel the second sheet 14 from the patient's skin with either hand while holding the needle with the other hand. Because the overlapping joint 18 completely divides the opening of the work-through area 20 into two separate portions, the sheets 12, 14 may be removed from around the needle.

In the exemplary arrangement shown in the drawings, the first sheet 12 has an outer peripheral edge that is generally rectangular, having a left edge 32a spaced from a right edge 32b, opposite spaced apart upper and lower edges 32c, 32d extending from the left edge to the right edge, and a first cutout area 34 along the left edge and spaced between the top edge and the bottom edge. The second sheet 14 also has an outer peripheral edge that is generally rectangular, having a left edge 36a spaced from a right edge 36b, opposite spaced apart upper and lower edges 36c, 36d extending from the left edge to the right edge, and a second cutout area 38 defined along the right edge and spaced between the top edge and the bottom edge. However, the sheets 12, 14 and the surgical drape 10 may be any preselected shape as may be desired for a particular application, such as round, oval, and/or other polygonal or non-geometric shapes, within the principles of the disclosure. In addition, where the work-through area 20 is defined by a removable area, the cut-outs 34 and 38 are replaced by tear-away areas of the respective sheets 12, 14 defined by frangible paths traversing the outer edges of the cut-outs 34, 38.

In an assembled condition ready for use, for example in a surgical procedure, the left edge 32a of the first sheet 12 partially overlaps and is releasably secured to the right edge 36b of the second sheet 14, forming the surgical drape 10 with the single outer peripheral edge 22. The first cutout or tear-away area 34 is aligned with the second cutout or tear-away area 38 to define the work-through area 20 in the central area of the surgical drape 10. The cutouts or tear-away areas 34, 38 may be formed separately in each sheet 12, 14 prior to joining the sheets along the overlapping joint 18 or the cutouts or tear-away areas 34, 38 may be formed after the sheets 12, 14 are joined along the overlapping joint 18. Without being bound to any particular size or shape, in one exemplary arrangement, each of the first and second sheets 12 may be between approximately 5 cm to 60 cm tall (i.e., from the top edge to the bottom edge), between approximately 5 cm to 60 cm wide (i.e., from the left edge to the right edge), the sheets may overlap a width of between approximately 1-2 cm to approximately 20-30 cm, and the work-through opening may be between approximately 2 cm in diameter to approximately 45 cm in diameter.

The sheets 12, 14 are secured together with the removable adhesive 16 sandwiched between the first and second sheets 12, 14 along the overlapping joint 18, such as in a first strip on one side of the work-through area 20 aligned with a second strip on an opposite side of the work-through area 20. The overlapping joint 18 has a length that extends across the surgical drape 10 in two portions 18a, 18b on opposites sides of the work-through area 20, such as in a straight line extending diametrically across the work-through area 20 from one edge of the surgical drape along the top edges 32c, 36c of the sheets 12, 14 to an opposite second edge of the surgical drape along the bottom edges 32d, 36d, thereby separating the surgical drape 10 into two separable portions, such as left and right halves defined by the sheets 12 and 14 that extend through the work-through area 20. The overlapping joint 18 preferably has a width that is less than the entire width of the drape between opposite left and right edges of the surgical drape as seen in the drawings. Preferably only a portion of each of the first and second sheets overlap 12, 14, such that a non-overlapping portion of the first sheet 12 extends in one direction from the overlapping joint 18, such as to the right in the figures, and a non-overlapping portion of the second sheet 14 extends in an opposite direction from the overlapping joint 18, such as to the left in the figures. In this arrangement, the overlapping joint 18 forms a relatively narrow strip extending through a central portion of the surgical drape 10. However, in other arrangements, the overlapping joint 18 may extend only part way across the surgical drape 10, for example, extending from only the outer peripheral edge 22 to the work-through area 20. In other arrangements, the overlapping joint 18 may have two portions that extend across the surgical drape 10 in an angularly offset arrangement rather than being aligned, the overlapping joint 18 may extend in a curved arrangement, and/or the overlapping joint 18 may extend in other shapes and arrangements capable of allowing the first sheet 12 to be separated from the second sheet 14 sufficiently to provide at least one path from the outer peripheral edge 22 to the work-through area 20 through which a needle, tube, or other surgical item may pass when removing the surgical drape 10 from a patient.

The work-through area 20 has a pre-defined size and shape and is spaced inwardly from the outer peripheral edge 22 of the surgical drape 10 such that the work-through area 20 is completely surrounded by at least some area of surgical drape material. The work-through area 20 may be centered in the area of the surgical drape 10, such as concentric with a geometric center point of the surgical drape 10, or the work-through area 20 may be offset from the center point. In some arrangements, the work-through area 20 is a pre-cut opening through one or both of the sheets 12, 14, and in other arrangements, the work-through area is a tear-away removable portion of the first and/or second sheets 12, 14 defined by a frangible path, such as a path of perforations, traversing a closed loop spaced inwardly from the outer peripheral edge 22 that pre-defines the size and shape of the work-through area 20. Although shown as circular in the drawings, the work-through area 20 may have other shapes, such as rectangular, square, oval, or other polygonal or non-specific shapes. The cutout or tear-away areas 34, 38 may be any shape necessary to form the pre-selected size and shape of the work-through area 20, such as for a particular suggested application of the drape. In the exemplary arrangement, each cutout or tear-away area 34, 38 is generally semi-circular to define a circular work-through area 20 in the surgical drape 10 when the first sheet 12 is secured to the second sheet 14 along the overlapping joint 18. Other sizes and shapes of cutouts may be used to define an opening of other preselected sizes and/or shapes.

The tab 30 is aligned with the overlapping joint 18 and disposed at or near the outer peripheral edge 22 of the surgical drape 10. In the exemplary arrangement, the tab 30 is formed by a portion of the first sheet 12 that does not overlap the second sheet 14 and/or that is not adhesively connected to the second sheet 14 at one end of the overlapping joint 18, for example, formed by strip along the top edge 32c of the first sheet 12 that is offset outwardly from the adjacent top edge 36c of the second sheet 14. The tab 30 may have other forms suitable for facilitating easy gripping by a user, such as a specially shaped portion that projects outwardly from top edge of the first sheet 12, a piece of material secured to a top side of the first sheet 12 and having a free end that is not connected to the first sheet, and/or an overlapping portion of one of the sheets 12, 14 that is not glued together at one of the ends of the overlapping joint 18. Optionally, a second tab 30a is located along the second section of the overlapping joint on the opposite side of the work-through area 20 as the first tab 30. For example, the second tab may be tab similar to the tab 30 and disposed at the opposite end of the overlapping joint 18 along the bottom edge 32d of the first sheet 12.

The removable areas 26a, 26b have predefined shapes and sizes defined by the shape and/or outline of the frangible paths 28a, 28b. The removable areas 26a, 26b may be provided in any shape or size desired for a particular suggested application and may completely surround the work-through area 20 or only partially surround the work-through area 20. In the exemplary arrangement, the first removable area 26a is adjacent the work-through area 20 in the form of a ring-shaped area of the sheets 12, 14 surrounding the work-through area 20 and defined by the first frangible path traversing a circular path spaced radially outwardly from a circular opening. The second removable area 26b is adjacent the first removable area 26a in the form of a second ring-shaped area of the sheets 12, 14 surrounding the first ring shaped area and defined by the second frangible path 28b traversing a circular path spaced radially outwardly from the first frangible path 28c. The frangible paths 28a, 28b are formed by, for example, perforations, die cuts, die stamped regions, pre-formed creases, or any other structure that makes the frangible paths weaker than surrounding areas so as to guide tearing of the sheet material along the frangible paths. The removable areas 26a, 26b may have other shapes, some examples of which include pie-shaped wedges, squares, or strips extending radially outwardly from the edge 24 of the work-through area 20. Further, two or more adjacent removable areas extending radially outwardly from the work-through area 20 may be defined by different patterns of frangible paths, for example forming multiple successive radial bands or layers of removable regions, whereby the size and/or shape of the work-through area 20 may be readily adapted for different applications by tearing along appropriate frangible paths and removing one or more of the removable areas from the sheets.

The adhesive 16 along the overlapping joint 18 is adapted to secure the first sheet 12 to the second sheet 14 under normal use during a surgical procedure and adapted to allow the first sheet 12 to be easily pulled away from the second sheet 14 without needing to rip or tear the surgical drape material. Preferably, the adhesive 16 is a removable pressure sensitive adhesive that forms a temporary bond and can be removed after substantial periods of time without having formed a permanent bond to the opposite sheet or adhesive carried by the opposite sheet. However, other types of removable adhesives that are sufficient to hold the first and second sheets 12, 14 together during normal storage and use of the surgical drape 10 and also allow the sheets 12, 14 to be separated by peeling release along the adhesive holding the overlapping joint 18 together may also be used, such as hot-melt adhesives, contact adhesives, and the like.

The adhesive 26 used for securing the surgical drape 10 to a patient is preferably a bio-compatible adhesive suitable for releasably securing the surgical drape to the skin of the patient. Preferably, areas of adhesive 26 on the back side of the surgical drape 10 are covered with a peel-away protective covering, such as waxed paper or plastic, to protect the adhesive 26 during storage and allow the adhesive 26 to be selectively exposed for securing the surgical drape 10 to a patient when selectively desired during a surgical procedure.

Each sheet 12, 14 is made of surgical drape material formed of one or more materials suitable for use as sterile surgical drapes, such as gauze, cloth, paper, plastic, foil, and/or various combinations thereof. Preferably, the surgical drape material is flexible and suitable for being adhered to the skin of a human patient and suitable for sterilization. Most preferably, the surgical drape material is approved for use as a surgical drape during some or all surgical procedures on humans by, for example, overseeing regulatory bodies, such as governmental agencies or industry regulatory groups. The sheets may be single ply or multi-ply and, without being bound to a particular dimension, may for example range in thickness from between approximately 50-100 mils to approximately 0.5-2 cm.

In one method of using the surgical drape 10, the user identifies how large of an opening is needed or desired as a work-through area 20 for the surgical procedure. If the work-through area 20 is defined by a pre-formed opening and is sufficiently large, then the peel-away protective covering may be removed from the areas of adhesive 26 on the back side, and the surgical drape 10 is adhered to the patient's skin with the aligned over the desired surgical region. If the work-through area 20 is defined by a tear-away area of the sheets 12,14, then the tear away area is removed from the surgical drape 10, for example, by tearing along the frangible path defining the work-through area 20 to define the opening. If the opening is not sufficiently large, the user may remove one or more of the pre-defined removable areas 26a, 26b, for example by tearing along one or more of the pre-defined frangible paths 28a, 28b, and thereafter adhering the surgical drape 10 to the patient. After the conclusion of the surgical procedure, the entire surgical drape 10 may be removed from the patient as a single piece. Alternatively, for example if the medical professional needs to hold a needle against the patient through the work-through area 20, the user may hold the needle with one hand and grasp the tab 30 with the other hand. The user then peels the first sheet 12 away from the second sheet 14 by pulling along the overlapping joint 18 and removes the first sheet 12 from the patient and around the needle. Subsequently, the user may grasp the second sheet 14 with either hand and then remove the second sheet from the patient around the needle. In this manner, the surgical drape 10 provides an adjustable work-through area in the form of a central opening, or fenestration, that can be adapted for different medical procedures and provides for easy removal of the surgical drape 10 with one hand without disturbing a tool, such as a needle and/or tube, that is secured to the patient through the central opening.

According to additional aspects, the surgical drape 10 may be provided in a pre-assembled form, as shown for example in FIG. 1, or the surgical drape 10 may be provided in an unassembled form, such as in the form of a kit. For example, the surgical drape 10 may be provided in the form of a kit by providing the first sheet 12, providing the second sheet 14, defining the work-through area 20 in at least one of the first and second sheets 12, 14, and providing the removable adhesive 16 for securing the first sheet 12 to the second sheet 14 along the overlapping joint 18. The work-through area is arranged to intersect the overlapping joint 18 when the first sheet 12 is secured to the second sheet 14 along the overlapping joint 18. The first and second cutout areas 34, 38 are located in the respective first and second sheets 12, 14 such that the cutouts define the work-through area 20 when the first sheet 12 is secured to the second sheet 14 along the overlapping joint 18. The removable areas 26a, 26b adjacent the work-through area 20 are defined by forming the frangible paths 28a, 28b in one or both of the first and second sheets 12, 14. The kit may be provided with each of the first and second sheets 12, 14 and the adhesive provided in unassembled form or partly assembled, and the surgical sheet may be subsequently assembled by, for example, the user or an intermediate assembler. The kit may include any or all of the various features described above in an unassembled form.

### INDUSTRIAL APPLICABILITY

In some aspects, the surgical drapes disclosed herein are useful for covering portions of a patient surrounding an incision or other penetration through the skin. However, the surgical drapes described herein are not limited to any particular exemplary uses and procedures detailed herein and may be used in any other manners or application as desired and would be understood by one of ordinary skill in the art.

Numerous modifications to the surgical drapes disclosed herein will be apparent to those skilled in the art in view of the foregoing description. Accordingly, this description is to be construed as illustrative only and is presented for the purpose of enabling those skilled in the art to make and use the invention and to teach the best mode of carrying out same. The exclusive rights to all modifications within the scope of the appended claims are reserved.

I claim:

1. A surgical drape comprising:
a first sheet comprising a flexible surgical drape material;
a second sheet comprising a flexible surgical drape material;
an overlapping joint comprising an area where the first sheet overlaps the second sheet;
a removable adhesive disposed along an overlapping joint between the first and second sheets, the removable adhesive securing the first sheet to the second sheet;
an outer peripheral edge of the surgical drape defined by the secured first and second sheets; and
a pre-defined work-through area defined in a central portion of the drape spaced inwardly from the outer peripheral edge;
wherein the overlapping joint intersects the pre-defined work-through area.

2. The surgical drape of claim 1, wherein the pre-defined work-through area comprises a pre-cut opening through the first and/or second sheets.

3. The surgical drape of claim 2, wherein the overlapping joint extends from an outer peripheral edge of the surgical drape to the opening.

4. The surgical drape of claim 3, further comprising a first removable portion of at least one of the first and second sheets adjacent the opening, the first removable portion defined by a first frangible path in one or both of the first and second sheets.

5. The surgical drape of claim 4, further comprising a tab defined by the first sheet and arranged to allow the first sheet to be separated from the second sheet.

6. The surgical drape of claim 5, wherein the tab is aligned with the overlapping joint at the outer peripheral edge of the surgical drape.

7. The surgical drape of claim 3, wherein the first sheet only partially overlaps the second sheet, and the overlapping joint extends from a first edge of the surgical drape to a second edge of the surgical drape and divides the opening into two separate portions.

8. The surgical drape of claim 4, further comprising a second removable portion defined by a second frangible path, wherein the first removable portion is adjacent the second removable portion.

9. The surgical drape of claim 8, wherein the first removable portion surrounds the opening, and the second removable portion surrounds the first removable portion.

10. The surgical drape of claim 9, wherein the first frangible path is in the shape of a circle surrounding and spaced radially from the opening, the second frangible path is in the shape of a circle surrounding and spaced from the first frangible path, and each frangible path is formed of perforations through the respective first or second sheets.

11. The surgical drape of claim 1, wherein the work-through opening comprises a pre-defined tear-away area of at least one of the first and second sheets defined by a frangible path traversing the work-through opening.

12. A surgical drape comprising:
a first sheet of surgical drape material;
a second sheet of surgical drape material partially overlapping the first sheet along an overlapping joint, wherein the first and second sheets define an outer peripheral edge of the surgical drape;
a removable adhesive disposed along the overlapping joint and engaging the first sheet and the second sheet; and
an pre-defined work-through area formed in at least one of the first and second sheets and spaced inwardly from the outer peripheral edge;
wherein the overlapping joint intersects the pre-defined work-through area.

13. The surgical drape of claim 11, wherein the overlapping joint is defined by a first edge of the first sheet that overlaps a second edge of the second sheet, and wherein the pre-defined work-through area is defined by a first cutout in the first sheet along the first edge and a second cutout in the second sheet along the second edge.

14. The surgical drape of claim 11, wherein the overlapping joint is defined by a first edge of the first sheet that overlaps a second edge of the second sheet, and wherein the pre-defined work-through area is defined by a first tear-away area defined by a frangible path traversing a closed loop.

15. The surgical drape of claim 11, further comprising a removable area defined in at least one of the first and second sheets adjacent the work-through area.

16. The surgical drape of claim 11, wherein the removable adhesive comprises a pressure sensitive adhesive.

17. The surgical drape of claim 11, further comprising a tab carried by the first sheet adjacent an end of the overlapping joint.

18. A method of providing a surgical drape comprising the steps:
providing a first sheet comprising a surgical drape material;
providing a second sheet comprising a surgical drape material;
defining a work-through area in at least one of the first and second sheets; and
providing a removable adhesive for securing the first sheet to the second sheet along an overlapping joint where the first sheet partially overlaps the second sheet;
wherein the work-through area is arranged to intersect the overlapping joint.

19. The method of claim 18, further comprising the step:
defining a removable area adjacent the work-through area in at least one of the first and second sheets with a frangible path in one or both of the sheets.

20. The method of claim 19, wherein the first sheet comprises a first cutout along a first edge, the second sheet comprises a second cutout along a second edge, and the first and second cutouts are located such that the cutouts define the work-through area when the first sheet is secured to the second sheet along the overlapping joint.

21. The method of claim 20, wherein the removable area comprises a first portion of a tear-away section partly surrounding the first cutout and a second portion of a tear-away section partly surrounding the second cutout, the first and second tear-away sections located to align with each other when the first sheet is secured to the second sheet along the overlapping joint.

22. The method of claim 21, wherein the first sheet defines a tab, the tab located to be adjacent the overlapping joint.
